(19) United States
(12) Patent Application Publication
(10) Pub. No.: US 2026/0073298 A1
(43) Pub. Date: Mar. 12, 2026

(54) TRAINED MODEL GENERATION METHOD, TRAINED MODEL GENERATION DEVICE, AND RECORDING MEDIUM

(71) Applicant: Panasonic Intellectual Property Management Co., Ltd., Osaka (JP)

(72) Inventors: Maho SHIOTANI, Osaka (JP); Shino IGUCHI, Kyoto (JP)

(21) Appl. No.: 19/257,772

(22) Filed: Jul. 2, 2025

Related U.S. Application Data
(63) Continuation of application No. PCT/JP2023/039969, filed on Nov. 7, 2023.
(60) Provisional application No. 63/438,819, filed on Jan. 13, 2023.

(30) Foreign Application Priority Data
Sep. 4, 2023 (JP) 2023-142898

(51) Int. Cl. G06N 20/00; G06V 30/416; G16H 10/60

(52) U.S. Cl. CPC G06N 20/00; G06V 30/416; G16H 10/60

(57) ABSTRACT

A trained model generation method includes: determining, per first period, whether a subject has an anomaly in a physical condition, based on a care record including text data; extracting, based on activity data on the subject in a second period that includes a plurality of first periods each being the first period, one or more features calculated for one or more first periods in which the subject is determined to have no anomaly in the physical condition, the one or more features being extracted from among one or more features per first period; and generating, through learning, a trained model that uses a feature of the subject as an input and outputs an anomaly score indicating a degree of an anomaly in the physical condition of the subject, the learning using the one or more features as input data, and using the anomaly score as training data.

### CROSS REFERENCE TO RELATED APPLICATIONS

This is a continuation application of PCT International Application No. PCT/JP2023/039969 filed on Nov. 7, 2023, designating the United States of America, which is based on and claims priority of Japanese Patent Application No. 2023-142898 filed on Sep. 4, 2023 and U.S. Provisional Patent Application No. 63/438819 filed on Jan. 13, 2023. The entire disclosures of the above-identified applications, including the specifications, drawings and claims are incorporated herein by reference in their entirety.

### FIELD

The present disclosure relates to a trained model generation method, a trained model generation device, and a recording medium.

### BACKGROUND

The 2025 problem in Japan is an aging society problem in which all eight million people belonging to what is called “Dankai no Sedai” (the baby boomer generation) will reach the ages of 75 or older, resulting in a quarter of the nation's population reaching the ages of 75 or older. This problem involves a problem of a labor shortage caused by increasing demands for medical and caregiving services.

Against this backdrop, the number of subjects of nursing and care who are looked after by health care workers and caregivers is increasing. As a result, a small change in a physical condition that may lead to an anomaly in physical condition may be overlooked. If a small change in a physical condition is overlooked, there is a risk that a subject may become more severely ill.

To deal with this, for example, Patent Literature (PTL) 1 discloses a technique of notifying an appropriate recipient of an anomaly in a monitored person when an anomaly in the monitored person is determined. Accordingly, an anomaly of the monitored person can be notified to an appropriate monitoring person in accordance with an anomaly state of the monitored person.

### CITATION LIST

### Patent Literature

PTL 1: WO 2018/116830

### SUMMARY

### Technical Problem

However, PTL 1 described above only discloses a technique of providing the notification in the case where vital information of the monitored person obtained from a sensor indicates an anomalous value, and thus the technique is not capable of detecting a small change in a physical condition that may lead to an anomaly in the monitored person, that is, a sign of an anomaly.

In view of the above, the present disclosure provides a trained model generation method, a trained model generation device, and a recording medium capable of generating a trained model that can assist in detecting a sign of an anomaly in a subject.

### Solution to Problem

A trained model generation method according to an aspect of the present disclosure includes: determining, per first period, whether a subject has an anomaly in a physical condition, based on a care record of the subject including text data, using a natural language processing model; obtaining activity data on the subject in a second period that includes a plurality of first periods each being the first period; calculating a feature per first period, based on the activity data obtained; extracting, based on a result of the determining, one or more features calculated for one or more first periods in which the subject is determined to have no anomaly in the physical condition, the one or more features being extracted from among a plurality of features calculated as the feature per first period; and generating, through supervised learning, a trained model that uses a feature of the subject as an input and outputs an anomaly score indicating a degree of an anomaly in the physical condition of the subject, the supervised learning using, as input data, the one or more features extracted, and using, as training data, the anomaly score corresponding to the one or more features extracted.

A trained model generation device according to an aspect of the present disclosure includes: a determiner that determines, per first period, whether a subject has an anomaly in a physical condition, based on a care record of the subject including text data, using a natural language processing model; an obtainer that obtains activity data on the subject in a second period that includes a plurality of first periods each being the first period; a calculator that calculates a feature per first period, based on the activity data obtained; an extractor that extracts, based on a result of the determination, one or more features calculated for one or more first periods in which the subject is determined to have no anomaly in the physical condition, the one or more features being extracted from among a plurality of features calculated as the feature per first period; and a generator that generates, through supervised learning, a trained model that uses a feature of the subject as an input and outputs an anomaly score indicating a degree of an anomaly in the physical condition of the subject, the supervised learning using, as input data, the one or more features extracted, and using, as training data, the anomaly score corresponding to the one or more features extracted.

A recording medium according to an aspect of the present disclosure is a non-transitory computer-readable recording medium having recorded thereon a program for causing a computer to execute the trained model generation method described above.

### Advantageous Effects

According to an aspect of the present disclosure, it is possible to implement a trained model generation method and the like capable of generating a trained model that can assist in detecting a sign of an anomaly in a subject.

### BRIEF DESCRIPTION OF DRAWINGS

These and other advantages and features will become apparent from the following description thereof taken in conjunction with the accompanying Drawings, by way of non-limiting examples of embodiments disclosed herein.

FIG. 1 is a diagram illustrating an example of a configuration of a physical condition detection system according to an embodiment.

FIG. 2 is a block diagram illustrating an example of a functional configuration of an information management server according to an embodiment.

FIG. 3 is a flowchart illustrating operation of generating a trained model by an information management server according to an embodiment.

FIG. 4 is a flowchart illustrating detailed operation of step S10 illustrated in FIG. 3.

FIG. 5 is a diagram showing an example of inputs and outputs of a natural language processing model according to an embodiment.

FIG. 6 is a flowchart illustrating detailed operation of step S20 illustrated in FIG. 3.

FIG. 7 is a diagram for describing training data according to an embodiment.

FIG. 8 is a diagram for describing how the training of a data augmented model proceeds according to an embodiment.

FIG. 9 is a diagram for describing how the training of a supervised model proceeds according to an embodiment.

FIG. 10A is a diagram illustrating a result of comparison in respiratory rate between actual data and expanded data.

FIG. 10B is a diagram illustrating a result of comparison in heart rate between actual data and expanded data.

FIG. 11 is a flowchart illustrating operation of detecting a sign of an anomaly in the physical condition of a subject in an information management server according to an embodiment.

FIG. 12 is a diagram for describing detection of a sign of an anomaly in the physical condition of a subject according to an embodiment.

FIG. 13 is a diagram showing an example of graded scores in five levels and conditions for the graded scores according to an embodiment.

FIG. 14A is a diagram illustrating a result of comparison in successful detection rate between a conventional method and the method according to the present disclosure.

FIG. 14B is a diagram illustrating a result of comparison in erroneous detection rate between a conventional method and the method according to the present disclosure.

### DESCRIPTION OF EMBODIMENTS

A trained model generation method according to a first aspect of the present disclosure includes: determining, per first period, whether a subject has an anomaly in a physical condition, based on a care record of the subject including text data, using a natural language processing model; obtaining activity data on the subject in a second period that includes a plurality of first periods each being the first period; calculating a feature per first period, based on the activity data obtained; extracting, based on a result of the determining, one or more features calculated for one or more first periods in which the subject is determined to have no anomaly in the physical condition, the one or more features being extracted from among a plurality of features calculated as the feature per first period; and generating, through supervised learning, a trained model that uses a feature of the subject as an input and outputs an anomaly score indicating a degree of an anomaly in the physical condition of the subject, the supervised learning using, as input data, the one or more features extracted, and using, as training data, the anomaly score corresponding to the one or more features extracted.

Accordingly, it is possible to obtain the anomaly score by inputting the plurality of features calculated from the activity data into the trained model generated through the supervised learning. With this anomaly score, it is possible to notice (detect) a sign of an anomaly in the physical condition. For example, in the case where a graded score for indicating a physical condition anomaly level of the subject in a graded manner is calculated based on the anomaly score, the sign of an anomaly in the physical condition of the subject can be noticed (detected) from the graded score. Thus, by the trained model generation method according to the present disclosure, it is possible to generate the trained model for detecting a sign of an anomaly (an anomaly in the physical condition) of the subject. Furthermore, it can be expected that using the trained model generated through the supervised learning improves an accuracy of the sign of an anomaly in the physical condition compared with the case of using a trained model generated through unsupervised learning.

Also, for example, a trained model generation method according to a second aspect is the trained model generation method according to the first aspect, and may further include training, using the one or more features extracted, a data augmented model that outputs a feature corresponding to a case where the subject has no anomaly in the physical condition, in which the one or more features used as the input data may include a feature generated by the data augmented model.

Accordingly, it is possible to automatically generate training data for performing the supervised learning, which can thus reduce or eliminate, for example, a user's task of making input on whether the subject is normal or has an anomaly.

Also, for example, a trained model generation method according to a third aspect is the trained model generation method according to the second aspect, in which in the calculating of the feature per first period, a plurality of features may be calculated per first period, and when a two-dimensional arrangement of the plurality of features calculated is referred to as a feature set, the data augmented model may output the feature set in which the plurality of features are two-dimensionally arranged.

Accordingly, it is possible to multiply a data set of the features that are two-dimensionally arranged, by n.

Also, for example, a trained model generation method according to a fourth aspect is the trained model generation method according to the second aspect or the third aspect, in which a data generative model may be used as the data augmented model.

Accordingly, with the data generative model, it is possible to effectively multiply the data set of the features that are two-dimensionally arranged, by n.

Also, for example, a trained model generation method according to a fifth aspect is the trained model generation method according to any one of the second through fourth aspects, in which one of a conditional generative adversarial network (CGAN), a variational autoencoder (VAE), an autoregressive model, or a diffusion model may be used as the data augmented model.

Accordingly, it is possible to generate the data augmented model using an existing model such as a CGAN model, a VAE model, an autoregressive model, or a diffusion model.

Also, for example, a trained model generation method according to a sixth aspect is the trained model generation method according to any one of the first through fifth aspects, and may further include: obtaining the care record of the subject; obtaining information indicating, in correspondence with the text data included in the care record, whether the subject has an anomaly in the physical condition; and generating the natural language processing model through supervised learning that uses, as input data, the text data obtained, and uses, as training data, the information obtained that indicates whether the subject has an anomaly in the physical condition.

Accordingly, by only obtaining the text data included in the care record, it is possible to automatically determine whether the subject has an anomaly in the physical condition at that time. For example, a user who nurses or cares for the subject need not determine whether the subject has an anomaly in the physical condition corresponding to the text data. Thus, it is possible to reduce a work load on the user.

Also, for example, a trained model generation method according to a seventh aspect is the trained model generation method according to any one of the first through sixth aspects, in which one of an anomaly detection with generative adversarial network (AnoGAN), a variational autoencoder (VAE), or a deep support vector data description (Deep SVDD) may be used as the trained model.

Accordingly, it is possible to generate the trained model using an existing model such as an AnoGAN model, a VAE model, or a Deep SVDD model.

Also, for example, a trained model generation method according to an eighth aspect is the trained model generation method according to any one of the first through seventh aspects, in which the activity data may include at least one of food intake, a respiratory rate, a heart rate, a body temperature, or an out-of-bed rate of the subject.

Accordingly, it is possible to generate the trained model for detecting a sign of an anomaly in the subject using at least one of the food intake, the respiratory rate, the heart rate, the body temperature, or the out-of-bed rate of the subject. Using the at least one of these makes it possible to detect a sign of an anomaly with high accuracy.

Also, for example, a trained model generation method according to a ninth aspect is the trained model generation method according to any one of the first through eighth aspects, in which the activity data may include at least two of food intake, a respiratory rate, a heart rate, a body temperature, or an out-of-bed rate of the subject.

Accordingly, it is possible to generate the trained model for detecting a sign of an anomaly in the subject using at least two of the food intake, the respiratory rate, the heart rate, the body temperature, or the out-of-bed rate of the subject. Using the at least two of these makes it possible to detect a sign of an anomaly with high accuracy.

A trained model generation device according to a tenth aspect of the present disclosure includes: a determiner that determines, per first period, whether a subject has an anomaly in a physical condition, based on a care record of the subject including text data, using a natural language processing model; an obtainer that obtains activity data on the subject in a second period that includes a plurality of first periods each being the first period; a calculator that calculates a feature per first period, based on the activity data obtained; an extractor that extracts, based on a result of the determination, one or more features calculated for one or more first periods in which the subject is determined to have no anomaly in the physical condition, the one or more features being extracted from among a plurality of features calculated as the feature per first period; and a generator that generates, through supervised learning, a trained model that uses a feature of the subject as an input and outputs an anomaly score indicating a degree of an anomaly in the physical condition of the subject, the supervised learning using, as input data, the one or more features extracted, and using, as training data, the anomaly score corresponding to the one or more features extracted. Also, a recording medium according to an eleventh aspect of the present disclosure is a non-transitory computer-readable recording medium having recorded thereon a program for causing a computer to execute the trained model generation method according to any one of the first through ninth aspects.

Accordingly, it is possible to produce the same advantageous effects as those produced by the trained model generation method described above.

Note that these general and specific aspects may be implemented using a system, a method, an integrated circuit, a computer program, or a non-transitory computer-readable recording medium such as a compact disc read-only memory (CD-ROM), or any combination of systems, methods, integrated circuits, computer programs, or recording media. The program(s) may be stored in a recording medium in advance or may be supplied to a recording medium via a wide area communication network including the Internet, for example.

Hereinafter, exemplary embodiments will be specifically described with reference to the drawings.

Note that each of the exemplary embodiments described below shows a general or specific example. The numerical values, constituent elements, the arrangement and connection of the constituent elements, steps, the processing order of the steps etc. shown in the following exemplary embodiments are mere examples, and therefore do not intend to limit the present disclosure. Among the constituent elements in the following exemplary embodiments, those not recited in any one of the independent claims will be described as optional constituent elements.

The drawings are represented schematically and are not necessarily precise illustrations. Thus, the scales, for example, are not necessarily consistent from drawing to drawing. In the drawings, constituent elements that are substantially the same are given the same reference signs, and redundant descriptions will be omitted or simplified.

In the present specification, numerical values and numerical value ranges do not express the strict meanings only, but also include substantially equivalent ranges, e.g., differences of several percent (or about 10%).

In the present specification, ordinal numerals such as “first” and “second” do not mean the number or order of constituent elements unless otherwise stated in particular. The ordinal numerals are used to avoid confusion of and distinguish between constituent elements of the same type.

### Embodiment

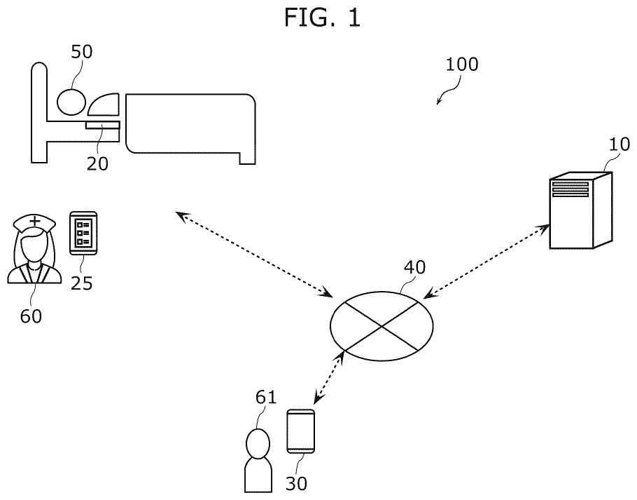

Hereinafter, a trained model generation method and the like according to the present embodiment will be described with reference to FIG. 1 through FIG. 14B.

### [1. Configuration of Physical Condition Detection System]

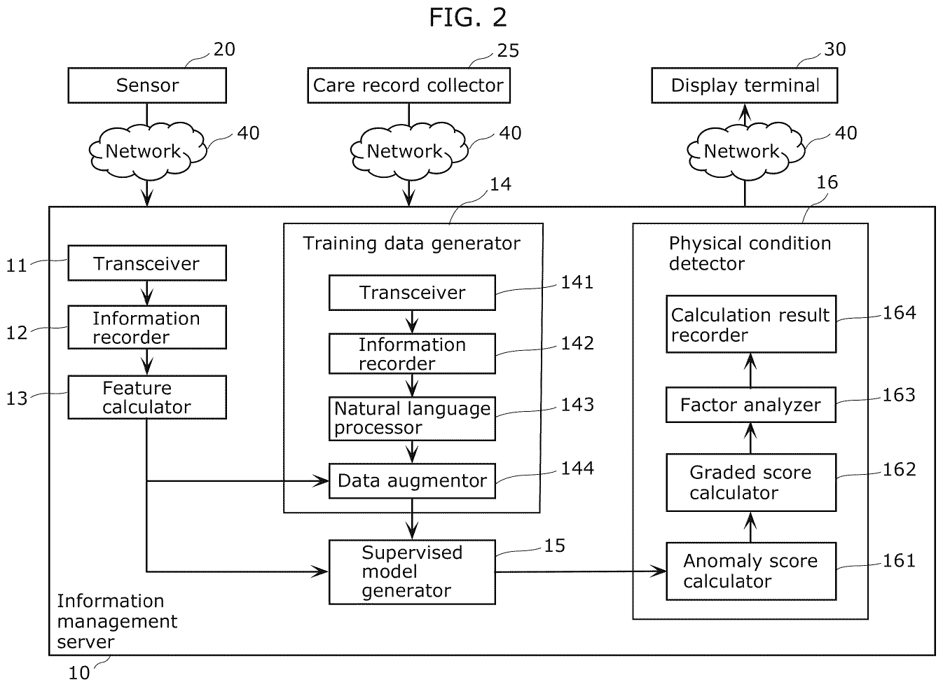

First, a configuration of a physical condition detection system including an information management server that executes the trained model generation method will be described with reference to FIG. 1 and FIG. 2. FIG. 1 is a diagram illustrating an example of a configuration of physical condition detection system 100 according to the present embodiment.

Physical condition detection system 100 according to the present embodiment is a system configured such that information management server 10 detects a small change in the physical condition of subject 50 of nursing or care that may lead to an anomaly in the physical condition of subject 50 (i.e., a sign of an anomaly in the physical condition).

As illustrated in FIG. 1, physical condition detection system 100 includes information management server 10, sensor 20, care record collector 25, and display terminal 30. These are connected communicably connected together via communication network 40. Communication network 40 may be a wired network, may be a wireless network, or may include both a wired network and a wireless network. FIG. 1 also illustrates subject 50 of nursing or care, user 60 who is an on-site staff member such as a health care worker who performs nursing or care of subject 50, and user 61 who is an on-site staff member such as a monitoring person who monitors subject 50 and can check display terminal 30.

Note that although FIG. 1 illustrates an example of the case where physical condition detection system 100 includes one sensor 20, this is not limiting, and it suffices if physical condition detection system 100 includes as many sensors 20 as subjects 50 of nursing or care.

Sensor 20 obtains activity data on subject 50 during a predetermined time period by sensing. The activity data includes at least one of a heart rate, a respiratory rate, in/out of bed, a body temperature, or a food intake. The activity data may include at least two of the heart rate, the respiratory rate, the in/out of bed, the body temperature, or the food intake. For example, sensor 20 may obtain data on the heart rate, the respiratory rate, a body motion, and the like (hereinafter, also referred to as sensor data) every second while subject 50 is in bed. Sensor 20 may further sense whether subject 50 is in or out of bed according to whether sensor 20 can sense the heart rate, the respiratory rate, the body motion, and the like.

Note that an interval at which the sensor data including the heart rate, the respiratory rate, the body motion, and the like is obtained is not limited to one second. The interval may be two seconds. The interval may be an interval in any unit so long as it enables sensing of changes in the sensor data of subject 50. Sensor 20 may further sense a life rhythm such as a sleep state according to whether sensor 20 can sense the heart rate, the respiratory rate, the body motion, and the like.

For example, sensor 20 may be a sensor device having a pressure sensor or the like and may be placed in a bed to sense subject 50 every second. In this case, for example, sensor 20 may output, every second, the value “1” indicating being out of bed as sensor data indicating that subject 50 is out of bed. For example, sensor 20 may output sensor data such as the respiratory rate of subject 50 every second.

Sensor 20 may be, for example, an image capturing device such as a camera and is provided such that sensor 20 can capture subject 50 who is in bed or subject 50 who is eating. The camera may be a thermal camera that detects the body temperature of subject 50 or may be a normal camera (e.g., a charge coupled device (CCD) camera). The food intake is an amount of food taken by subject 50 in the morning, afternoon, night, or the like. The food intake may be obtained by performing image analysis on an image. Note that the body temperature and the food intake may be obtained as inputs into care record collector 25 by user 60.

Note that the following will mainly describe the case where the activity data include the respiratory rate and the heart rate.

Care record collector 25 collects care records from user 60. The care records are each a record of details of nursing or care of subject 50 by user 60. Each care record includes text data on a free description of a condition and the like of subject 50 when user 60 performs the nursing or care of subject 50.

Care record collector 25 includes an input unit that receives a care record from user 60 and a display that displays a screen on which a care record is input. The input unit is, for example, but not limited to, a touch panel, a keyboard, or a sound collecting device (e.g., a microphone). The input unit is, for example, but not limited to, a display device such as a liquid crystal display. Care record collector 25 may be a mobile terminal device such as a smartphone or a tablet, and may be a stationary terminal device such as a personal computer (PC).

Information management server 10 is implemented using, for example, a computer including a processor (a microprocessor), a memory, a communication interface, and the like. Information management server 10 may be configured to operate with a part of the configuration of information management server 10 included in a cloud server. Information management server 10 generates a trained model for detecting a small change in a physical condition of subject 50 that may lead to an anomaly in the physical condition of subject 50 (i.e., a sign of an anomaly in the physical condition) and uses the generated trained model to detect the small change in the physical condition of subject 50 that may lead to the anomaly in the physical condition of subject 50.

FIG. 2 is a block diagram illustrating an example of a functional configuration of information management server 10 according to the present embodiment.

As illustrated in FIG. 2, information management server 10 includes transceiver 11, information recorder 12, feature calculator 13, training data generator 14, supervised model generator 15, and physical condition detector 16. Transceiver 11, information recorder 12, feature calculator 13, training data generator 14, and supervised model generator 15 constitute a trained model generation device. Note that the trained model generation device may be implemented as a stand-alone device.

Transceiver 11 includes, for example, a communication interface and transmits and receives various types of information to and from sensor 20 or display terminal 30 via communication network 40. Transceiver 11, for example, obtains activity data including the respiratory rate and the heart rate of subject 50 during a predetermined time period. Here, the activity data may include, for example, at least the respiratory rate and the heart rate among the body temperature, food intake, the respiratory rate, the heart rate, and an out-of-bed rate of subject 50 during the predetermined time period. Further, transceiver 11 outputs the graded score calculated by physical condition detector 16 to a terminal possessed by user 61 such as a monitoring person who monitors subject 50.

In the present embodiment, transceiver 11 obtains the sensor data such as the heart rate, the respiratory rate, and the body motion per second while subject 50 is in bed, from sensor 20 via communication network 40 at predetermined intervals, for example, every minute. In this manner, transceiver 11 obtains activity data that includes the sensor data and is obtained on-site every day, via communication network 40. Transceiver 11 also obtains activity data in a second period, which is longer than a first period (e.g., one day) described later. The second period is, for example, a time necessary to obtain a desired number of activity data items to be used for training a data augmented model described later. For example, the second period is several months.

Transceiver 11 also transmits a graded score calculated by physical condition detector 16 to display terminal 30 via communication network 40. Note that transceiver 11 may transmit information for a display that is presented on a user interface of display terminal 30 to cause user 61 to handle an anomaly in the physical condition of subject 50, such as a graded score display, a vital fluctuation graph display, or a risk group display described later. Transceiver 11 may also obtain at least one of the body temperature or the food intake of subject 50 that are included in the care record. Transceiver 11 is an example of an obtainer.

Information recorder 12 records information transmitted and received by transceiver 11. Information recorder 12 is a recording medium capable of recording information and includes, for example, a rewritable, nonvolatile memory such as a hard disk drive and a solid state drive. Note that information recorder 12 may record a plurality of features calculated by feature calculator 13.

Feature calculator 13 includes, for example, a computer including a memory and a processor (microprocessor). With the processor executing a control program stored in the memory, feature calculator 13 implements a function of calculating the plurality of features. Feature calculator 13 calculates the plurality of features based on the activity data including respiratory rates and heart rates of subject 50 that are obtained by transceiver 11. For example, feature calculator 13 obtains sensor data for a time period including a target date and time of detecting the physical condition from activity data obtained by transceiver 11 or recorded on information recorder 12 and calculates hourly features for each type of sensor data such as the respiratory rate. Feature calculator 13 is an example of a calculator.

Here, feature calculator 13 may calculate, for example, at least a mean value and a maximum value of respiratory rates of subject 50 and a mean value and a maximum value of heart rates of subject 50, as a plurality of hourly features. In the present embodiment, feature calculator 13 calculates, from at least the respiratory rates and the heart rates, the mean values and the maximum values of the respiratory rates and the heart rates as the plurality of features, from among mean values, maximum values, standard deviations, skewnesses, kurtoses, and impulse factors of the respiratory rates, difference data on the respiratory rates, the heart rates, and difference data on the heart rates. Here, each of the impulse factors is obtained by subtracting the corresponding mean value from the corresponding maximum value. In this manner, feature calculator 13 performs statistical processing and the like on the activity data to calculate the plurality of features.

More specifically, feature calculator 13 calculates, for example, respiratory-rate-related features and heart-rate-related features of subject 50 on an hourly basis. For example, feature calculator 13 obtains sensor data indicating respiratory rates of subject 50 within a time period including a target date and time of detecting the physical condition from activity data recorded on information recorder 12 or sensor data obtained from sensor 20 and calculates hourly statistical features for the time period.

In more detail, feature calculator 13 obtains, for example, respiratory rate data on respiratory rates not being zero during a given hour from the activity data and calculates, as the statistical features, a mean value, a maximum value, a minimum value, a standard deviation, a skewness, a kurtosis, an impulse factor, and the like during the hour from the obtained respiratory rate data. Here, the impulse factor can be calculated from a difference between the maximum value and the mean value (maximum value-mean value) of the respiratory rate data for the hour. Feature calculator 13 also calculates, as the statistical features, a mean value, a maximum value, a minimum value, a standard deviation, a skewness, a kurtosis, an impulse factor, and the like during the hour from the difference data on the obtained respiratory rate data. The difference data on the obtained respiratory rate data is data indicating, for example, a difference between a respiratory rate at time point t and a respiratory rate at a time point t+1, which is one second after time point t, that is, differences between items of the respiratory rate data on a per-second basis. Note that it suffices if feature calculator 13 calculates, as the statistical features, at least the mean value and the maximum value during the hour from the obtained respiratory rate data.

For example, feature calculator 13 also obtains heart rate data indicating heart rates of subject 50 within a time period including a target date and time of detecting the physical condition from the activity data recorded on information recorder 12 or the sensor data obtained from sensor 20 and calculates hourly statistical features for the time period.

Here, feature calculator 13 obtains, for example, heart rate data on heart rates not being zero during a given hour from the activity data and calculates, as the statistical features, a mean value, a maximum value, a minimum value, a standard deviation, a skewness, a kurtosis, an impulse factor, and the like during the hour from the obtained heart rate data. Feature calculator 13 also calculates, as the statistical features, a mean value, a maximum value, a minimum value, a standard deviation, a skewness, a kurtosis, an impulse factor, and the like during the hour from difference data on the obtained heart rate data. The difference data on the obtained heart rate data is data indicating, for example, a difference between a heart rate at time point t and a heart rate at a time point t+1, which is one second after time point t, that is, differences between items of the heart rate data on a per-second basis, as with the difference data on the respiratory rate data. Note that it suffices if feature calculator 13 calculates, as the statistical features, at least the mean value and the maximum value during the hour from the obtained heart rate data.

Note that feature calculator 13 may calculate one of the food intake, the out-of-bed rate, or the body temperature of subject 50 as one of the plurality of features.

For example, feature calculator 13 may calculate the food intake of subject 50 from care records included in the activity data, as one of the plurality of features. In this case, it suffices if feature calculator 13 calculates a total amount of food intakes in one day in the past from the care records and next calculates a total sum of food intakes within a time period including a target date and time of detecting the physical condition. Here, in the case where target dates and times are set in morning, afternoon, and night time periods, it suffices if feature calculator 13 calculates, for example, a total sum of food intakes during a period from the morning of a previous day of the target date of detecting the physical condition to the morning of the target date, a period from the afternoon of the previous day to the afternoon of the target date, and a period from the night of the previous day to the night of the target date.

For example, feature calculator 13 may also calculate an out-of-bed rate from the activity data obtained by transceiver 11 and recorded on information recorder 12, as one of the plurality of features. In this case, it suffices if feature calculator 13 obtains in-or-out-of-bed data indicating whether subject 50 is in or out of bed within a time period including a target date and time of detecting the physical condition from the activity data recorded on information recorder 12 or the sensor data obtained from sensor 20 and calculates an hourly out-of-bed rate for the time period. In more detail, feature calculator 13 can calculate an out-of-bed rate during a given hour by, for example, counting up the number of values “1” indicating being out of bed during the hour and dividing the number by a total number during the hour (i.e., a total of the number of values “1” indicating being out of bed and the number of values “0” indicating being in bed during the hour).

Alternatively, feature calculator 13 may calculate, for example, the body temperature of subject 50 from care records included in the activity data, as one of the plurality of features. In this case, it suffices if feature calculator 13 calculates a body temperature (e.g., a mean body temperature) for one day in the past from the care records and next calculates a body temperature (e.g., a mean body temperature) for a time period including a target date and time of detecting the physical condition. Here, in the case where target dates and times are set in morning, afternoon, and night time periods, it suffices if feature calculator 13 calculates, for example, body temperatures during a period from the morning of a previous day of the target date of detecting the physical condition to the morning of the target date, a period from the afternoon of the previous day to the afternoon of the target date, and a period from the night of the previous day to the night of the target date.

Training data generator 14 includes, for example, a computer including a memory and a processor (microprocessor). With the processor executing a control program stored in the memory, training data generator 14 implements a function of generating training data. Training data generator 14 generates the training data to be used by supervised model generator 15 to generate a supervised model. Training data generator 14 includes transceiver 141, information recorder 142, natural language processor 143, and data augmentor 144.

Transceiver 141 includes, for example, a communication interface and transmits and receives various types of information to and from care record collector 25 via communication network 40. Transceiver 141 obtains, for example, care records of subject 50 during a predetermined time period. Here, the care records include text data indicating a condition or the like of subject 50 during the predetermined time period.

In the present embodiment, transceiver 141 obtains, for example, a care record including details of nursing or care of subject 50 by user 60 being an on-site staff member as illustrated in FIG. 1 and text data indicating a condition or the like of subject 50 from care record collector 25 via communication network 40 on an hourly or daily basis. In this manner, transceiver 141 obtains a care record that includes the text data and is obtained on-site every day, via communication network 40.

Information recorder 142 records information transmitted and received by transceiver 141. Information recorder 142 is a recording medium capable of recording information and includes, for example, a rewritable, nonvolatile memory such as a hard disk drive and a solid state drive. Note that information recorder 142 may record a natural language processing model to be used by natural language processor 143, a data augmented model to be used by data augmentor 144, and a feature set augmented (multiplied by n) by data augmentor 144.

Note that an interval at which transceiver 141 obtains a care record is not limited to an hour or a day.

Natural language processor 143 determines, per first period (e.g., an hour or a day), whether subject 50 has an anomaly in the physical condition, based on text data included in a care record of subject 50 using the natural language processing model. The natural language processing model is a model that outputs, in response to an input of text data on subject 50 per first period, whether subject 50 has an anomaly in the physical condition during the first period. That is, natural language processor 143 outputs, from the text data included in the care record, during what period an anomaly in the physical condition of subject 50 occurs and during what period no anomaly in the physical condition of subject 50 occurs. An identified period during which no anomaly in the physical condition occurs (e.g., a normal day) is used to generate training data for training an augmented model in data augmentor 144.

In the present embodiment, natural language processor 143 generates the natural language processing model through supervised learning. Note that natural language processing model may be generated in advance and recorded on information recorder 142. That is, natural language processor 143 need not have a function of generating the natural language processing model.

As the natural language processing model, an existing model that supports a language spoken in a facility where the system is introduced (e.g., a care facility, a medical facility) is used. An example of the natural language processing model to be used is, but not limited to, one of UTH-BERT, a BERT pretrained in Japanese (a Japanese version of BERT), or a Robustly Optimized BERT Pretraining Approach (ROBERTa).

Note that, in the present embodiment, a Japanese version of BERT is used as the natural language processing model. For example, the Japanese version of BERT is generated through training using the data from “Wikipedia Cirrussearch” as of Aug. 31, 2020 (about 17 million sentences) with a task of masking words and predicting the hidden words. A method of importing the data is, but not limited to, using “BertForSequenceClassification.from_pretrained(cl-tohoku/bert-bas e-japanese)”, “AutoTokenizer.from_pretrained(cl-tohoku/bert-base-japanese)”, or the like. As a model structure of the Japanese version of BERT, a structure used in a typical BERT model is adopted. For example, the structure has, for example, “12 layers, hidden layers having 768 dimensions, and 12 attention heads”.

As seen from the above, training data generator 14 is configured to be capable of automatically determining whether subject 50 has an anomaly in the physical condition from text data by natural language processing. Accordingly, manual determination (manual labeling) of whether subject 50 has an anomaly in the physical condition is dispensed with. Thus, it is possible to reduce an on-site burden of performing a determination task for generating a supervised learning model on, for example, user 60. Natural language processor 143 is an example of a determiner.

Data augmentor 144 uses the data augmented model to generate features (e.g., a feature set) of the case where subject 50 has no anomaly. The data augmented model receives a noise vector or the like as input data and outputs the features of the case where subject 50 has no anomaly. The output features are used when supervised model generator 15 trains the supervised model. Data augmentor 144 can automatically generate a feature set necessary to perform the supervised learning. For example, in the case where a complex, high-performance supervised model is generated, a large number of training data items are needed, and data augmentor 144 can easily generate such a large number of training data items.

In the present embodiment, data augmentor 144 generates the data augmented model through the supervised learning. Note that data augmented model may be generated in advance and recorded on information recorder 142. That is, data augmentor 144 need not have a function of generating the data augmented model.

As the data augmented model, a data generative model is used, and in the present embodiment, an image generative model is used as an example of the data generative model. An example of the data augmented model to be used is, but not limited to, one of a conditional generative adversarial network (CGAN), a variational autoencoder (VAE), an autoregressive model, or a diffusion model.

Supervised model generator 15 generates a model that is trained with a feature set including a plurality of features (i.e., normal features of subject 50). More specifically, supervised model generator 15 performs the supervised learning using the feature set to generate a supervised model trained with normal features in the feature set including the plurality of features. When receiving a plurality of features of subject 50, the supervised model outputs an anomaly score indicating a degree of an anomaly in the physical condition of subject 50. Supervised model generator 15 will be described in detail later. The generated supervised model is an example of a trained model, and supervised model generator 15 is an example of a generator.

Supervised model generator 15 includes, for example, a computer that includes a memory and a processor (microprocessor). Supervised model generator 15 implements various functions with the processor executing a control program stored in the memory.

As the trained model, the supervised learning model is used. An example of the trained model is, but not limited to, one of an anomaly detection with generative adversarial network (AnoGAN), a variational autoencoder (VAE), or a deep support vector data description (Deep SVDD).

Physical condition detector 16 is implemented using, for example, a computer including a processor (microprocessor), a memory, and a communication interface. Physical condition detector 16 implements various functions with the processor executing a control program stored in the memory. Physical condition detector 16 detects an anomaly in the physical condition of subject 50 using the supervised model generated by supervised model generator 15 and the plurality of features calculated by feature calculator 13.

Physical condition detector 16 includes anomaly score calculator 161, graded score calculator 162, factor analyzer 163, and calculation result recorder 164.

Anomaly score calculator 161 inputs the plurality of features calculated by feature calculator 13 into the trained model (supervised model) generated through the supervised learning to obtain an anomaly score indicating a degree of an anomaly in the physical condition per predetermined time period.

In the present embodiment, anomaly score calculator 161 inputs, into the trained model generated by supervised model generator 15, a plurality of hourly features on a target date of detecting the physical condition of subject 50 that are calculated by feature calculator 13. Anomaly score calculator 161 records hourly anomaly scores on the target date of detection of the physical condition of subject 50 that are calculated, on calculation result recorder 164.

Based on the anomaly score calculated by anomaly score calculator 161, graded score calculator 162 calculates a graded score that indicates a physical condition anomaly level of subject 50 in a graded manner.

In the present embodiment, graded score calculator 162 calculates a daily anomaly score mean value from hourly anomaly scores of a target date of detecting the physical condition that are recorded on calculation result recorder 164 or calculated by anomaly score calculator 161. Likewise, graded score calculator 162 calculates daily anomaly score mean values on a previous day of the target date of detecting the physical condition and on the day before the previous day, from hourly anomaly scores on the previous day of the target date and on the day before the previous day that are recorded on calculation result recorder 164. Graded score calculator 162 totalizes the daily anomaly score means on the target date, the previous day, and the day before the previous day, thus calculating a three-day total score. Note that the three-day total score is an example of a total score used in a calculation method for calculating a graded score with high accuracy, and this is not limiting. It suffices if a period for calculating the total score ranges from one day to five days.

Graded score calculator 162 calculates threshold values for graded scores (may also be referred to as graded threshold values) from a three-day total score group for about 90 days in the past from the target date recorded on calculation result recorder 164. More specifically, graded score calculator 162 calculates the graded threshold values by calculating a mean and a standard deviation of the three-day total score group for about 90 days in the past.

Graded score calculator 162 outputs a value of the calculated graded score to calculation result recorder 164. For example, in the case where the graded scores are in five levels, graded score calculator 162 may further output the calculated graded score to display terminal 30 via communication network 40 when the value of the calculated graded score is from one to three.

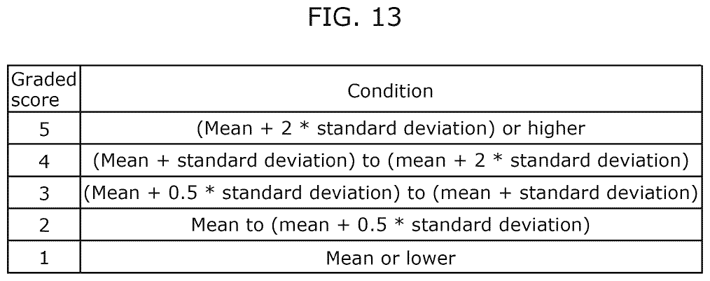

Processing by graded score calculator 162 will be described later with reference to FIG. 13.

Factor analyzer 163 performs factor analysis when the graded score is greater than or equal to a predetermined value to analyze, for each of elements included in the activity data, whether the element is a factor for the graded score being greater than or equal to the predetermined value. Here, the elements include, for example, food intake, the respiratory rate, the heart rate, the body temperature, or an out-of-bed rate of subject 50 during the predetermined time period. In addition, the predetermined value is a value at which handling a sign of an anomaly in a physical condition is needed. For example, in the case where the graded scores are in five levels, the predetermined value may be determined to be four or five, in the case where the graded scores are in three levels, the predetermined value may be determined to be three, and in the case where the graded scores are in two levels, the predetermined value may be determined to be two.

In the present embodiment, in the case where the graded score calculated by graded score calculator 162 is four or five, factor analyzer 163 performs a factor analysis on elements including heart rates, respiratory rates, out-of-bed rates, food intakes, and body temperatures, which are included in activity data used to calculate features. In the case where the activity data used to calculate features includes only the heart rates and the respiratory rates, it suffices if the factor analysis is performed on elements including the heart rates and the respiratory rates.

For example, factor analyzer 163 converts a plurality of features of each element in an entire time period used to calculate a graded score into data of a plurality of daily features of the element to calculate mean values and standard deviations in the entire time period used to calculate the graded score. In the present embodiment, factor analyzer 163 converts a plurality of features of each element in three days into data of a plurality of daily features of the element to calculate mean values and standard deviations of the element in the three days.

Factor analyzer 163 then makes an analysis showing that the element does not form a factor when Expression 1 shown below is established, and makes an analysis showing that the element forms a factor when Expression 1 shown below is not established.

$$( Mean value - 2 * standard deviation ) \leq ( feature of the element at target date and time ) \leq ( mean value + 2 * standard deviation ) \qquad ( Expression 1 )$$

Note that Expression 1 uses a nature of standard deviation that 95.45% of all data items are distributed within the range that is twice as much as mean value±standard deviation.

Factor analyzer 163 outputs the graded score and the element analyzed to be a factor by the factor analysis to calculation result recorder 164. Factor analyzer 163 may also output the graded score and the element analyzed to be a factor by the factor analysis to display terminal 30 via communication network 40.

Calculation result recorder 164 is a recording medium capable of recording a calculation result and includes, for example, a rewritable, nonvolatile memory such as a hard disk drive and a solid state drive. In the present embodiment, calculation result recorder 164 records, as the calculation result, the anomaly score calculated by anomaly score calculator 161, the graded score calculated by graded score calculator 162, and the like. Note that calculation result recorder 164 may record a factor analyzed by factor analyzer 163, as the calculation result.

Display terminal 30 is implemented using a computer including, for example, a processor (a microprocessor), memory, a communication interface, a user interface, and the like. Display terminal 30 is a terminal possessed by user 61 such as the monitoring person who monitors subject 50, and is, for example, a mobile terminal device such as a tablet or a smartphone. Display terminal 30 may be a mobile computer or a stationary computer (a stationary terminal device) connected to a display device.

In the present embodiment, display terminal 30 can be checked by user 61 such as the monitoring person who monitors subject 50. Display terminal 30 is connected to communication network 40, and when, for example, a graded score is obtained from information management server 10, causes the user interface to show a screen for user 61 to handle an anomaly in the physical condition of subject 50. The user interface can cause a display device to show a screen according to, for example, an input from user 61.

### [2. Operation of Information Management Server]

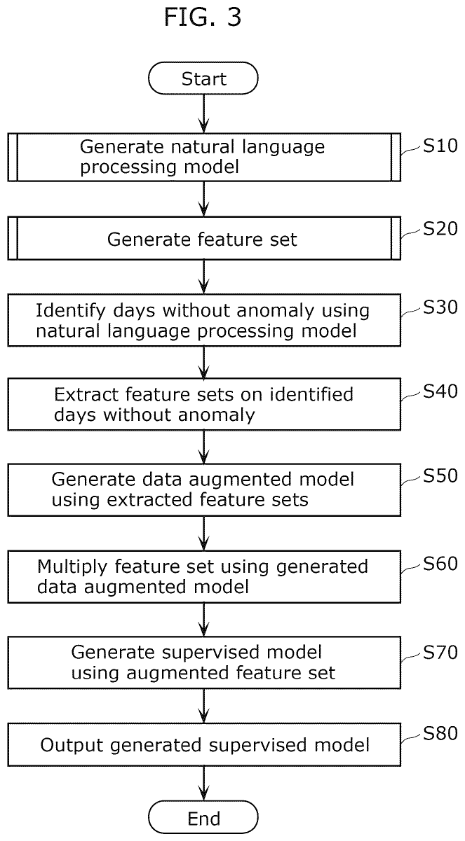

Subsequently, operation by information management server 10 configured as described above will be described with reference to FIG. 3 to FIG. 14B. First, operation of generating the trained model (supervised model) for detecting a sign of an anomaly in the physical condition of subject 50 will be described with reference to FIG. 3 to FIG. 10B.

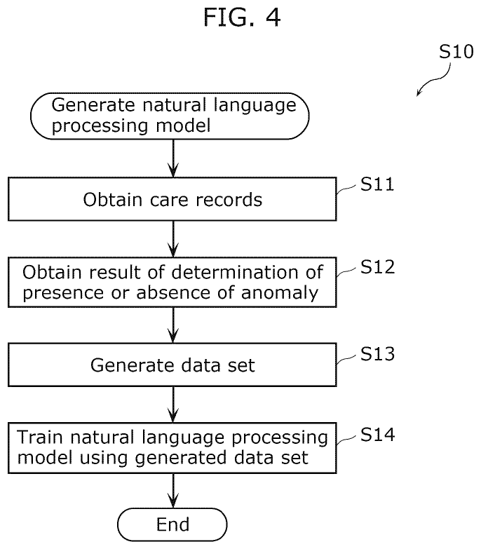

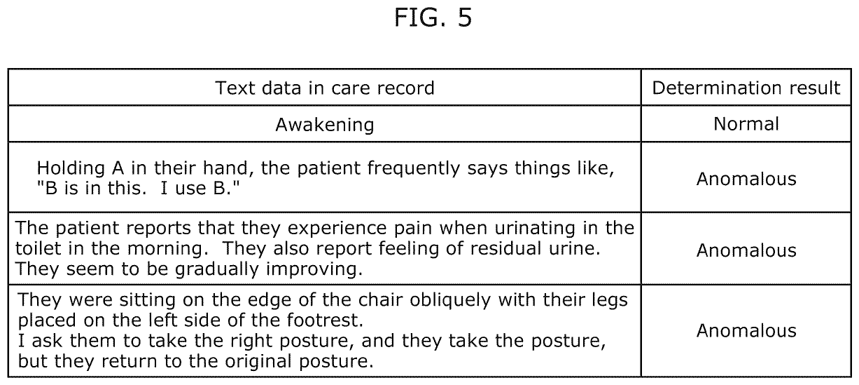

FIG. 3 is a flowchart illustrating operation of generating the trained model (the trained model generation method) by information management server 10 according to the present embodiment. Note that FIG. 3 illustrates, as an example, a flowchart including the case where the natural language processing model and the data augmented model are generated. FIG. 4 is a flowchart illustrating detailed operation of step S10 (the trained model generation method) illustrated in FIG. 3. FIG. 5 is a diagram showing an example of inputs and outputs of the natural language processing model according to the present embodiment.

As illustrated in FIG. 3, training data generator 14 first generates the natural language processing model (S10).

As illustrated in FIG. 4, transceiver 141 of training data generator 14 obtains care records of subject 50 (S11). The care records each include text data on a free description of a condition and the like of subject 50 when user 60 performs nursing or care of subject 50. Transceiver 141 obtains the text data items in the care records from care record collector 25 through communication. For example, transceiver 141 obtains text data items such as text data items in care records shown in FIG. 5. Examples of the text data include information indicating an action of subject 50 such as “awakening”, what subject 50 says, information indicating a subjective state of subject 50, and information on an attitude of subject 50. Transceiver 141 records the text data items on information recorder 142.

Next, transceiver 141 obtains a result of determination of presence or absence of an anomaly (an anomaly in the physical condition) for each text data item (S12). Transceiver 141 obtains the result of the determination for each text data item from care record collector 25 through communication. The presence or absence of an anomaly in the physical condition may be determined by, for example, user 60. Transceiver 141 records the results of the determination on information recorder 142 in association with the text data items.

Next, based on the text data items and the results of the determination, natural language processor 143 generates a data set for training (training data) necessary to execute the supervised learning (S13). In the data set, the text data items and the results of the determination are associated with each other.

Natural language processor 143 trains the natural language processing model using the generated data set (S14). Natural language processor 143 trains the natural language processing model through supervised machine learning using the data set. Specifically, natural language processor 143 trains the natural language processing model through supervised machine learning in which the text data items are used as input data and the results of the determination are used as training data (labeled data). Natural language processor 143 records the generated natural language processing model on information recorder 142.

The natural language processing model generated in this manner can serve as a model specific to subject 50. That is, a natural language processing model is provided for each subject 50.

Referring to FIG. 3 again, next, feature calculator 13 generates a feature set based on activity data obtained by transceiver 11 (S20).

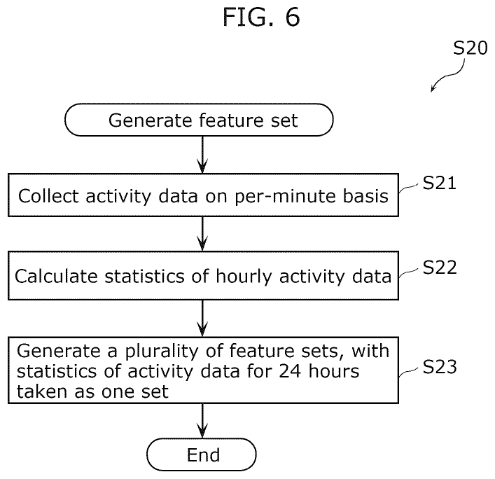

FIG. 6 is a flowchart illustrating detailed operation of step S20 (the trained model generation method) illustrated in FIG. 3.

As illustrated in FIG. 6, transceiver 11 collects activity data on a per-minute basis from sensor 20 through communication (S21). Transceiver 11 obtains sensor data including a heart rate, a respiratory rate, a body motion, or the like, every minute.

Next, feature calculator 13 calculates one or more statistics of hourly activity data (S22). Feature calculator 13 performs statistical processing and the like on heart rates and respiratory rates for an hour to calculate one or more statistics. Feature calculator 13 also performs statistical processing and the like on results of in/out of bed for an hour to calculate one or more statistics including an out-of-bed rate. The calculated statistic is an example of a feature.

Next, feature calculator 13 generates a plurality of feature sets (feature sets for a plurality of days), with statistics of activity data for 24 hours taken as one set (S23). Feature calculator 13 takes statistics of heart rates and respiratory rates for 24 hours and in/out of bed statuses for 24 hours as one set to be used as training data for the data augmented model.

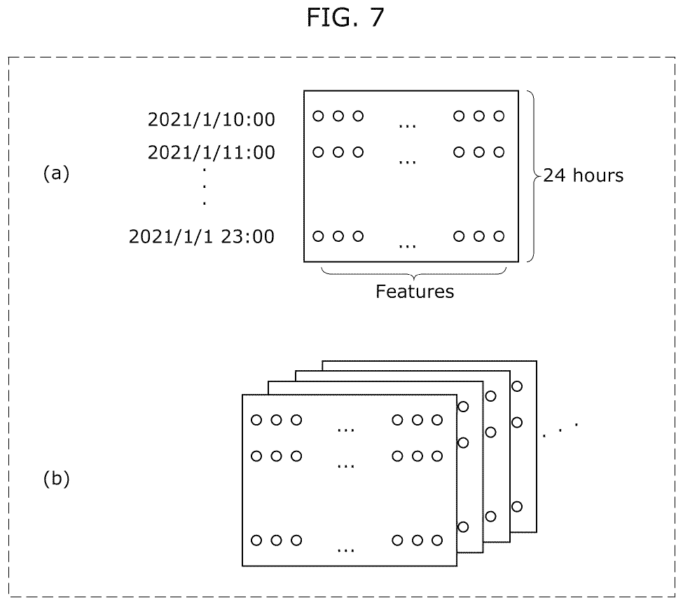

FIG. 7 is a diagram for describing the training data according to the present embodiment.

In FIG. 7, (a) is a diagram schematically illustrating a feature set for 24 hours (one set). The feature set is, for example, data in which features for 24 hours are two-dimensionally arranged.

In FIG. 7, (b) is a diagram illustrating the plurality of feature sets. Feature calculator 13 generates a plurality of feature sets each for 24 hours. By generating a feature set on a daily basis, feature calculator 13 generates the plurality of feature sets.

Feature calculator 13 outputs the plurality of generated feature sets to data augmentor 144.

Referring to FIG. 3 again, next, natural language processor 143 uses the natural language processing model to identify days on which subject 50 has no anomaly (S30). For example, natural language processor 143 identifies days (or dates and times) where subject 50 has no anomaly, from among a plurality of days on which activity data from which feature calculator 13 generates the plurality of feature sets is obtained from the care records recorded on information recorder 142. Here, having no anomaly may mean that there are no anomalies at all, may mean that the number of times an anomaly is determined in one day is less than or equal to a predetermined number of times, or may mean that only a minor level anomaly is detected. Natural language processor 143 functions as the determiner.

Natural language processor 143 inputs text data on each of the plurality of days into the natural language processing model and obtains presence or absence of an anomaly in the physical condition as an output of the natural language processing model. For example, as shown in FIG. 5, natural language processor 143 obtains a result of determination as to whether subject 50 is normal or has an anomaly for each text data item included in the care records. The example in FIG. 5 shows an example in which the text data item “awakening” is determined to be normal and the other text data items are determined to be abnormal. Note that in the case where there are a plurality of text data items in one day, a result of the determination is obtained for each of the text data items (for example, every hour).

Based on the output of the natural language processing model, natural language processor 143 identifies days on which subject 50 is normal, and outputs information indicating the identified days on which subject 50 is normal, to data augmentor 144.

Referring to FIG. 3 again, next, data augmentor 144 extracts feature sets on days that are identified by natural language processor 143 and on which subject 50 has no anomaly (days on which subject 50 is determined to be normal), from among the plurality of feature sets obtained from feature calculator 13 (S40). It can also be said that data augmentor 144 extracts one or more features (one or more feature sets) for one or more first periods in which subject 50 is determined to have no anomaly in the physical condition, from among the plurality of feature sets (plurality of features) calculated as feature sets on a daily (24 hours, an example of the first period) basis. That is, the extracted feature sets include only features that are generated from activity data on days on which subject 50 is determined to have no anomaly in the physical condition (normal days). The number of the extracted feature sets is assumed to be two or more. Data augmentor 144 functions as an extractor.

Note that, in the case where natural language processor 143 makes the determination as to whether subject 50 is normal or has an anomaly every hour, data augmentor 144 may extract, in step S40, feature sets on hours in which subject 50 has no anomaly.

Next, data augmentor 144 uses the two or more extracted feature sets to generate the data augmented model (S50). Data augmentor 144 uses the feature sets on the normal days extracted in step S40 to train, through machine learning, the data augmented model for generating data likely to be seen a normal day. Data augmentor 144 trains the data augmented model through a supervised machine learning in which a noise vector or the like is used as input data and “1 (real)” or “0 (fake)” are used as training data.

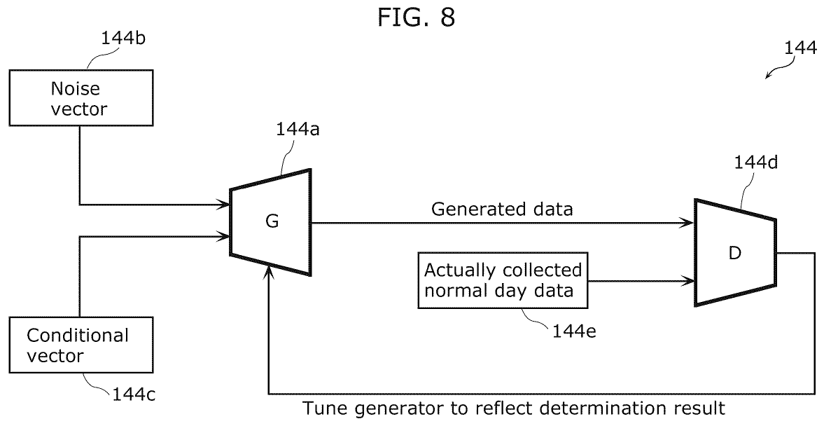

FIG. 8 is a diagram for describing how the training of the data augmented model proceeds according to the present embodiment. With reference to FIG. 8, model training using a CGAN will be described. Note that, as a training process for the data augmented model, various types of known training processes for GAN are adoptable.

As illustrated in FIG. 8, the data augmented model includes generator 144a (denoted as “G” in FIG. 8) and discriminator 144d (denoted as “D” in FIG. 8). For example, the data augmented model includes a neural network serving as generator 144a and a neural network serving as discriminator 144d.

Generator 144a receives, as inputs, noise vector 144b (also referred to as a random vector or a latent variable) and conditional vector 144c (also referred to as a label) and generates Fake data including a feature set for one set (data generated by the data augmented model, “Generated data” in FIG. 8). The Fake data is, for example, data having values close to those of the feature set illustrated in (a) in FIG. 7. The Fake data is data in which a plurality of features for one day (features as many as those shown in (a) in FIG. 7) are two-dimensionally arranged. When “0” is assumed to be normal and “1” is assumed to be abnormal, a vector representing “0” is input as conditional vector 144c every time because the data augmented model generates normal data in the present embodiment.

When discriminator 144d receives the Fake data generated by generator 144a, discriminator 144d determines whether the Fake data is real (Real data) or fake (synthetic data) (authenticity). When determining that the Fake data is Real data, discriminator 144d outputs “1”, and when determining that the Fake data is not Real data, discriminator 144d outputs “0”.

For example, normal day data 144e that is actually collected (i.e., training data) and data generated by generator 144a are input into discriminator 144d, which outputs “1” or “0” in accordance with a difference between the two data items. By making generator 144a reflect a result of determination by discriminator 144d (e.g., the reflection through backpropagation, etc.), parameters of generator 144a are updated. Then, data generated by updated generator 144a and normal day data 144e are input into discriminator 144d, and discriminator 144d outputs “1” or “0” in accordance with a difference between the two data items. By making discriminator 144d reflect a result of determination by discriminator 144d (e.g., the reflection through backpropagation, etc.), parameters of discriminator 144d are updated.

Note that normal day data 144e and the data generated by generator 144a are each data in which the same number of features are two-dimensionally arranged.

As an accuracy of the Fake data generated by generator 144a improves, a training accuracy of discriminator 144d that discriminates the Fake data improves. When discriminator 144d becomes capable of discriminating the authenticity with high accuracy, a training accuracy of generator 144a improves. In this manner, in the CGAN, generator 144a and discriminator 144d are made to compete with each other while alternately updated, thereby learning features of normal data. Accordingly, generator 144a becomes capable of outputting an accurate feature set corresponding to a time when subject 50 is normal, in response to inputs of noise vector 144b and conditional vector 144c, and discriminator 144d becomes capable of determining fake data (e.g., data on a time when subject 50 has an anomaly) with high accuracy.

The data augmented model generated in this manner can serve as a model specific to subject 50. That is, there are as many data augmented models as subjects 50.

Referring to FIG. 3 again, data augmentor 144 uses the generated data augmented model to multiply (by n) a feature set, a result of which is to be used as training data to train the supervised model (S60). Data augmentor 144 can produce a large number of feature sets only by changing noise vector 144b input into generator 144a. Since data augmentor 144 has been trained with the feature sets on the normal days, a feature set including values close to the feature set calculated from the activity data on the normal days of subject 50 can be generated in step S60. Data augmentor 144 may record the generated feature set on information recorder 142.

Next, supervised model generator 15 uses the augmented feature set to generate the supervised model (S70). Supervised model generator 15 trains, through machine learning, the supervised model that receives the feature set of subject 50 as input data and outputs an anomaly score of subject 50. Supervised model generator 15 trains the supervised model through a supervised learning in which a noise vector is used as input data and anomaly scores are used as training data. The anomaly scores take multiple values from 0 to 1, both inclusive. Alternatively, the anomaly scores may be binary, normal “0” or abnormal “1”. Note that supervised model generator 15 may further train the supervised model using the feature sets extracted in step S40.

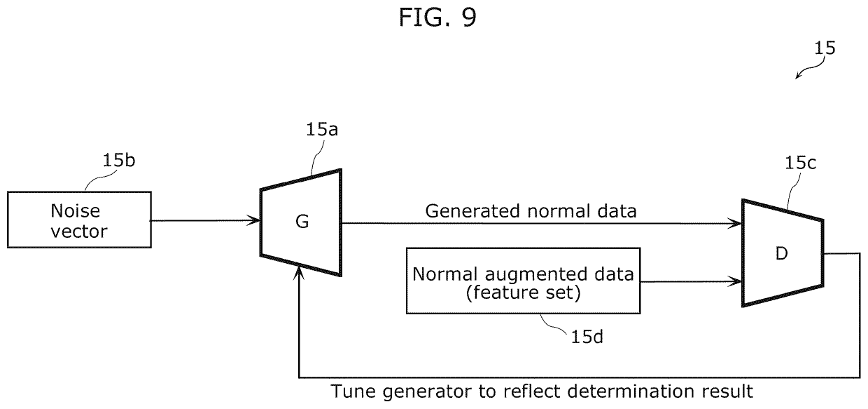

FIG. 9 is a diagram for describing how the training of the supervised model proceeds according to the present embodiment. With reference to FIG. 9, model training using an AnoGAN will be described. Note that, as a training process for the supervised model, various types of known training processes for GAN are adoptable.

As illustrated in FIG. 9, the supervised model includes generator 15a (denoted as “G” in FIG. 9) and discriminator 15c (denoted as “D” in FIG. 9). For example, the supervised model includes a neural network serving as generator 15a and a neural network serving as discriminator 15c.

Generator 15a receives, as inputs, noise vector 15b (also referred to as a random vector or a latent variable) and generates Fake data including a feature set for one set (“Generated normal data” in FIG. 9). The Fake data is, for example, data having values similar to those of the feature set illustrated in (a) in FIG. 7. The Fake data is data in which a plurality of features for one day (features as many as those shown in (a) in FIG. 7) are two-dimensionally arranged.

When discriminator 15c receives the Fake data generated by generator 15a, discriminator 15c determines whether the Fake data is real (Real data item) or fake (a synthetic data item) (authenticity). Discriminator 15c outputs a probability that the Fake data is Real data (e.g., a value ranging from 0 to 1, both inclusive).

For example, normal augmented data 15d (a feature set) (i.e., training data) and data generated by generator 15a are input into discriminator 15c, which outputs a value ranging from 0 to 1, both inclusive, in accordance with a difference between the two data items. Normal augmented data 15d and the data generated by generator 15a are each data in which the same number of features are two-dimensionally arranged.

By making generator 15a reflect a result of determination by discriminator 15c (e.g., the reflection through backpropagation, etc.), parameters of generator 15a are updated. Then, normal data generated by updated generator 15a and normal augmented data 15d are input into discriminator 15c, and discriminator 15c outputs a value ranging from 0 to 1, both inclusive, in accordance with a difference between the two data items. By making discriminator 15c reflect a result of determination by discriminator 15c (e.g., the reflection through backpropagation, etc.) , parameters of discriminator 15c are updated.

As an accuracy of the Fake data generated by generator 15a improves, a training accuracy of discriminator 15c that discriminates the Fake data improves. When discriminator 15c becomes capable of discriminating the authenticity with high accuracy, a training accuracy of generator 15a improves. In this manner, in the AnoGAN, generator 15a and discriminator 15c are made to compete with each other while alternately updated, thereby learning features of normal data. Accordingly, generator 15a becomes capable of outputting an accurate feature set corresponding to a time when subject 50 is normal, in response to an input of noise vector 15b, and discriminator 15c becomes capable of determining fake data (e.g., data on a time when subject 50 has an anomaly) with high accuracy.

The supervised model generated in this manner can serve as a model specific to subject 50. That is, there are as many supervised models as subjects 50.

Referring to FIG. 3 again, next, supervised model generator 15 outputs the generated supervised model to anomaly score calculator 161 (S80). Supervised model generator 15 may record the generated supervised model on a recorder (not illustrated) of information management server 10.

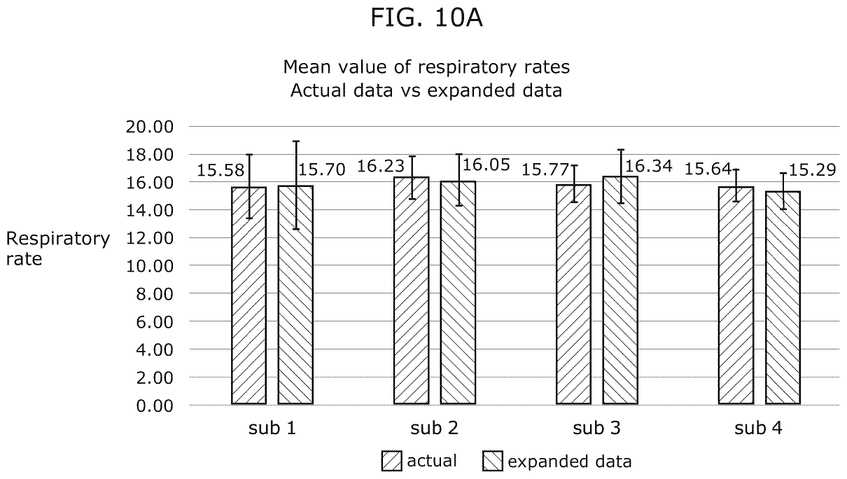

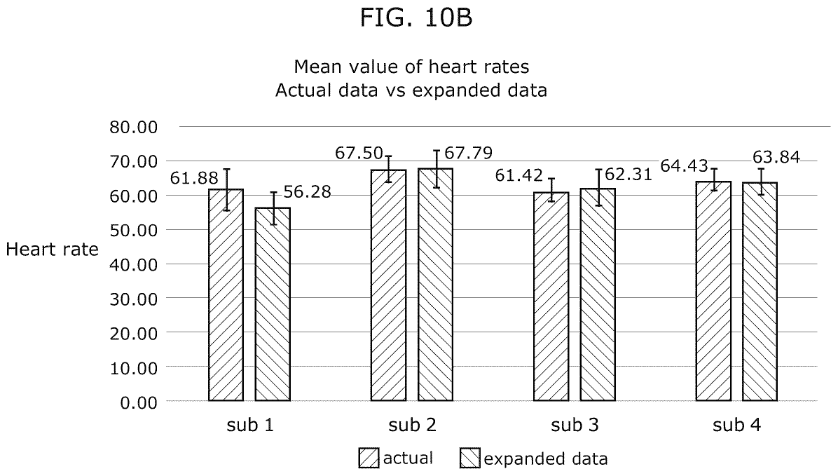

Here, with reference to FIG. 10A and FIG. 10B, the accuracy of the feature set produced through the multiplication in step S60 (expanded data) will be described. FIG. 10A is a diagram illustrating a result of comparison in respiratory rate between actual data and the expanded data. FIG. 10A illustrates results of comparison in mean value of respiratory rates of four subjects 50 including sub1 to sub4 (an example of the feature) between actual data and the expanded data. FIG. 10B is a diagram illustrating a result of comparison in heart rate between actual data and the expanded data. FIG. 10B illustrates results of comparison in mean value of heart rates of four subjects 50 including sub1 to sub4 (an example of the feature) between actual data and the expanded data. The actual data indicates values calculated based on sensor data from sensor 20, and the expanded data indicates the Fake data generated by the data augmented model. Note that the vertical axis in FIG. 10A represents respiratory rate for one minute, and the vertical axis in FIG. 10B represents heart rate for one minute.

As illustrated in FIG. 10A and FIG. 10B, the mean values of the actual data and the expanded data are almost the same in both respiratory rate and heart rate. Thus, it is understood that the data augmented model generated in the above manner successfully generates Fake data items that are close to the actual data items on normal days.

Although the above describes the example of multiplying a feature set using the data augmented model, this is not limiting. For example, the feature set need not be multiplied in the case where the number of the feature sets extracted in step S40 is greater than or equal to a number of feature sets that is enough to perform machine learning. In this case, in step S70, the supervised model may be generated using only the feature sets extracted in step S40.

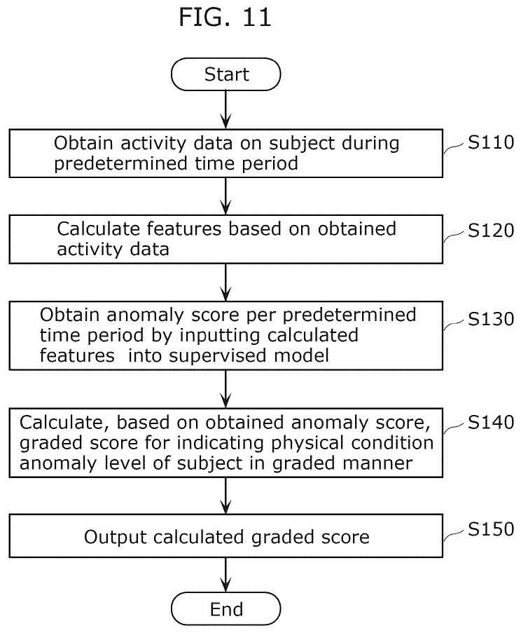

Subsequently, with reference to FIG. 11 to FIG. 14B, operation of detecting a sign of an anomaly in the physical condition of subject 50 using the supervised model generated as above will be described.

FIG. 11 is a flowchart illustrating the operation of detecting a sign of an anomaly in the physical condition of subject 50 in information management server 10 according to the present embodiment.

As illustrated in FIG. 11, transceiver 11 obtains activity data on subject 50 (e.g., activity data including respiratory rates and heart rates of subject 50) during a predetermined time period (S110).

Next, feature calculator 13 calculates features based on the activity data obtained in step S110 (S120). Feature calculator 13 calculates, for example, hourly features (e.g., a plurality of hourly features). For example, feature calculator 13 may calculate a plurality of hourly features on a target date of detecting the physical condition of subject 50 based on activity data including at least respiratory rates and heart rates of subject 50 obtained by transceiver 11.

Next, anomaly score calculator 161 obtains an anomaly score per predetermined time period by inputting the features calculated in step S120 into the supervised model that is pretrained (S130). Anomaly score calculator 161 calculates hourly anomaly scores from, for example, the plurality of hourly features. Anomaly score calculator 161 obtains anomaly scores each indicating a degree of an anomaly in the physical condition per hour in a predetermined time period including the target date, by inputting the features calculated by feature calculator 13 into the supervised model generated by supervised model generator 15.

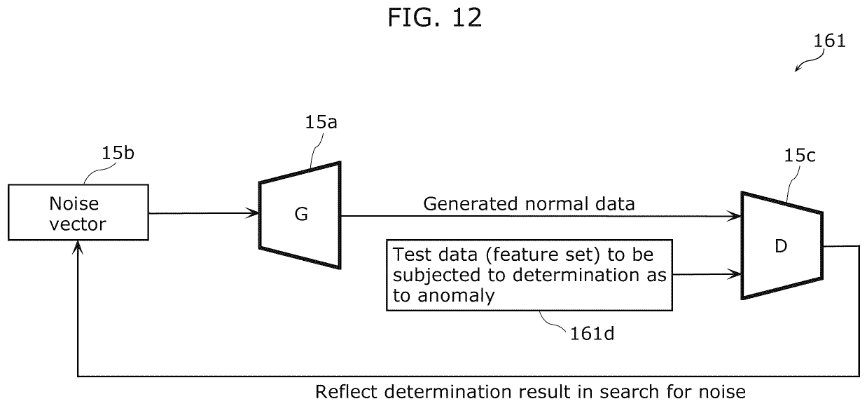

FIG. 12 is a diagram for describing the detection of a sign of an anomaly in the physical condition of subject 50 according to the present embodiment. Test data 161d is data on which the detection about an anomaly in the physical condition of subject 50 is performed using the supervised learning model. That is, test data 161d may be data on a time when an anomaly in the physical condition occurs or may be data on a time when no anomaly in the physical condition occurs.

As illustrated in FIG. 12, when discriminator 15c receives the normal data (data that looks like being normal) generated by generator 15a and test data 161d (a feature set) to be subjected to determination as to whether an anomaly in the physical condition of subject 50 occurs, discriminator 15c outputs a value ranging from 0 to 1, both inclusive, as a result of the determination in accordance with a difference between the two data items. In the case where test data 161d is data on a time when no anomaly in the physical condition occurs, discriminator 15c outputs a value close to zero. In the case where test data 161d is data on a time when an anomaly in the physical condition occurs, discriminator 15c outputs a value close to one.

A result of the determination by discriminator 15c is reflected in a search for noise, and thus noise vector 15b is determined. Generator 15a receives determined noise vector 15b as an input to generate new normal data. When discriminator 15c receives the new normal data (data that looks like being normal) generated by generator 15a and test data 161d (a feature set) to be subjected to determination as to whether an anomaly in the physical condition of subject 50 occurs, discriminator 15c outputs a value ranging from 0 to 1, both inclusive, as a result of the determination in accordance with a difference between the two data items. Here, since generator 15a has been trained to generate normal data, generator 15a generates normal data even when noise vector 15b being input is changed. That is, in the case where test data 161d is data on a time when no anomaly in the physical condition occurs, discriminator 15c constantly outputs a value close to zero. In the case where test data 161d is data on a time when an anomaly in the physical condition occurs, discriminator 15c constantly outputs a value close to one. Accordingly, discriminator 15c is capable of outputting, as the anomaly score, a probability of whether test data 161d is data on a time when an anomaly in the physical condition occurs.

Referring to FIG. 11 again, next, graded score calculator 162 calculates, based on the anomaly score obtained in step S130, a graded score that indicates a physical condition anomaly level of subject 50 in a graded manner (S140). For example, graded score calculator 162 calculates a daily mean value of anomaly scores from the hourly anomaly scores. In the present embodiment, graded score calculator 162 calculates a daily anomaly score mean value from the hourly anomaly scores in the predetermined time period including the target date of detecting the physical condition of subject 50.

Graded score calculator 162 also calculates the graded threshold values by calculating a mean and a standard deviation of past anomaly scores before the target date (e.g., anomaly scores for roughly past 90 days).

FIG. 13 is a diagram showing an example of graded scores in five levels and conditions for the graded scores according to the present embodiment.

As shown in FIG. 13, for example, when calculating the graded scores in five levels, graded score calculator 162 can calculate the threshold values from a mean and a standard deviation. For example, according to the conditions shown in FIG. 13, a threshold value for a graded score of one is the mean, and threshold values for a graded score of two are between the mean and a value that is subtraction of a value being half the standard deviation from the mean.

Graded score calculator 162 then applies the calculated graded threshold values to the mean value of the anomaly scores on the target date, thus calculating the graded score. More specifically, graded score calculator 162 calculates a value of the graded score by subjecting the mean value of the anomaly scores on the target date to determination using the threshold values calculated under the conditions shown in FIG. 13.

Graded score calculator 162 may further check whether the graded score calculated in step S140 indicates a value of four or five, that is, whether a value indicating an anomaly in the physical condition is calculated. In the case where the graded score is the value of four or five, factor analyzer 163 may perform a factor analysis on elements including heart rates, respiratory rates, out-of-bed rates, body temperatures, and food intakes, which are included in the activity data used to calculate the features.

Next, graded score calculator 162 outputs the graded score calculated in step S140 (S150). In the case where an element has been analyzed to be a factor by the factor analysis by factor analyzer 163, graded score calculator 162 may output in step S150 the graded score and the element analyzed to be a factor by the factor analysis.

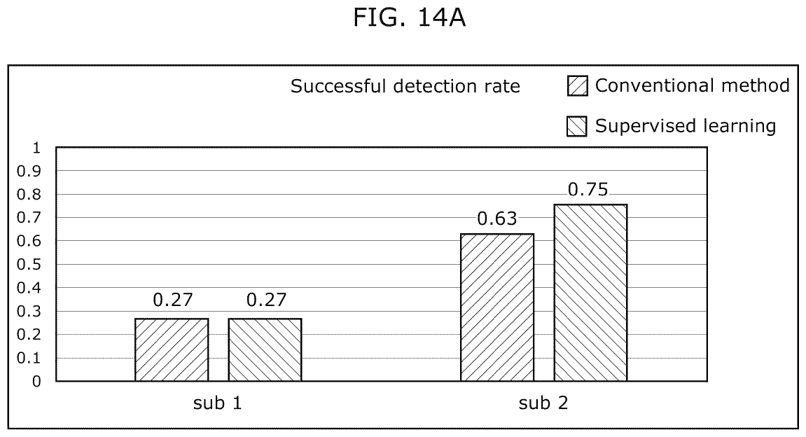

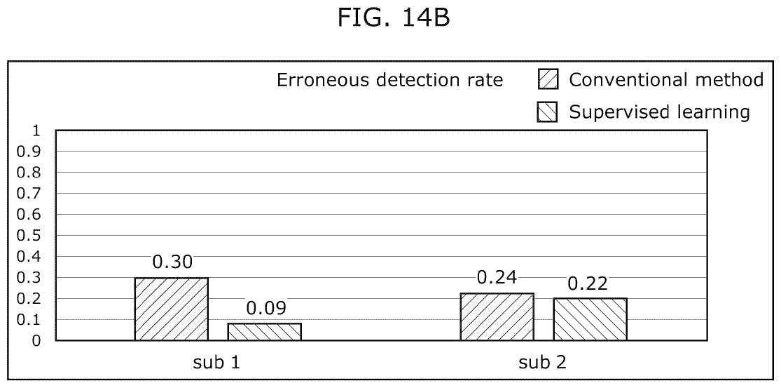

Here, with reference to FIG. 14A and FIG. 14B, an accuracy of the supervised model generated in step S70 will be described. FIG. 14A is a diagram illustrating a result of comparison in successful detection rate between a conventional method and the method according to the present disclosure. FIG. 14A illustrates a result of comparison in successful detection rate of anomaly in physical condition for two subjects 50 including sub1 and sub2. FIG. 14B is a diagram illustrating a result of comparison in erroneous detection rate between the conventional method and the method according to the present disclosure. FIG. 14B illustrates a result of comparison in erroneous detection rate of anomaly in physical condition for two subjects 50 including sub1 and sub2. The vertical axis in FIG. 14A represents the successful detection rate (0 to 1), and the vertical axis in FIG. 14B represents the erroneous detection rate (0 to 1). Note that the conventional method means that an unsupervised model generated through unsupervised learning is used as the trained model used by anomaly score calculator 161.

As illustrated in FIG. 14A, using the supervised learning improves the successful detection rate of anomaly in physical condition to be equivalent to or better than that of the conventional method.

As illustrated in FIG. 14B, using the supervised learning decreases the erroneous detection rate of anomaly in physical condition compared with that of the conventional method.

As illustrated in FIG. 14A and FIG. 14B, it is understood that using the supervised model improves at least one of the successful detection rate or the erroneous detection rate.

### Other Embodiments

Although the trained model generation method and so on according to one or more aspects have been described above based on an embodiment, the present disclosure is not limited to the embodiment. The present disclosure may also include forms achieved by making various modifications to the above embodiment that can be conceived by those skilled in the art, as well as forms achieved by combining constituent elements in different embodiments, without materially departing from the spirit of the present disclosure.

For example, in the above embodiment, the data augmented model is trained with only the feature set corresponding to the time when subject 50 is normal. However, this is not limiting. The data augmented model may be trained with only a feature set corresponding to a time when subject 50 has an anomaly.

In the above embodiment, each of the constituent elements may be configured in the form of an exclusive hardware product, or may be implemented by executing a software program suitable for the constituent element. Each of the constituent elements may be implemented by a program executor, such as a central processing unit (CPU) or a processor, reading and executing the software program recorded on a recording medium such as a hard disk or semiconductor memory.

The processing order of executing the steps shown in the flowcharts is a mere illustration for specifically describing the present disclosure, and thus may be an order other than the shown order. Also, one or more of the steps may be executed simultaneously (in parallel) with another step, or may be steps that are not executed.

The divisions of the functional blocks shown in the block diagrams are mere examples, and thus a plurality of functional blocks may be implemented as a single functional block, or a single functional block may be divided into a plurality of functional blocks, or one or more functions may be moved to another functional block. Also, the functions of a plurality of functional blocks having similar functions may be processed by a single hardware or software product in a parallelized or time-divided manner.

The information management server according to the above embodiment may be implemented as a single device or may be implemented by a plurality of devices. When the information management server is implemented by a plurality of devices, the constituent elements included in the information management server may be assigned to the plurality of devices in any manner. When the information management server is implemented by a plurality of devices, the method of communication between the plurality of devices is not particularly limited; the communication may be wireless communication or may be wired communication. Wireless communication and wired communication may be combined for communication between the devices.

Each of the constituent elements described in the above embodiment may be implemented in the form of a software product, or may be typically implemented as a large-scale integrated (LSI) circuit, which is an integrated circuit (IC). These may take the form of individual chips, or may be partially or entirely packaged into a single chip. Although the term “LSI” is used here, other names, such as IC, system LSI, super LSI, and ultra LSI may be used, depending on the level of integration. Furthermore, the manner in which the circuit integration is achieved is not limited to LSI, and it is also possible to use a dedicated circuit (a general-purpose circuit that executes a dedicated program) or a general-purpose processor. A field programmable gate array (FPGA) that allows for programming after the manufacture of an LSI circuit, or a reconfigurable processor that allows for reconfiguration of the connection and the setting of circuit cells inside an LSI circuit may be employed. Furthermore, when advancement in semiconductor technology or derivatives of other technologies brings forth a circuit integration technology which replaces LSI, it will be appreciated that such a circuit integration technology may be used to integrate the constituent elements.

A system LSI circuit is a super-multifunctional LSI circuit manufactured with a plurality of processing units integrated on a single chip, and is specifically a computer system including a microprocessor, read-only memory (ROM), and random-access memory (RAM), for example. A computer program is stored in the ROM. The system LSI circuit achieves its function as a result of the microprocessor operating according to the computer program.

An aspect of the present disclosure may be a computer program that causes a computer to execute each characteristic step included in the trained model generation method illustrated in any one of FIG. 3, FIG. 4, FIG. 6, or FIG. 11.

For example, the program may be a program to be executed by a computer. An aspect of the present disclosure may be a non-transitory computer-readable recording medium having such a program recorded thereon. For example, such a program may be recorded on a recording medium and distributed. For example, by installing the distributed program in a device that includes another processor and causing the processor to execute the program, it is possible to cause the device to perform each of the processes described above.

### INDUSTRIAL APPLICABILITY

The present disclosure is applicable to a trained model generation method for generating a trained model that can assist in detecting a small change in the physical condition of a subject that may lead to an anomaly in the physical condition of the subject.

1. A trained model generation method comprising:
determining, per first period, whether a subject has an anomaly in a physical condition, based on a care record of the subject including text data, using a natural language processing model;
obtaining activity data on the subject in a second period that includes a plurality of first periods each being the first period;
calculating a feature per first period, based on the activity data obtained;
extracting, based on a result of the determining, one or more features calculated for one or more first periods in which the subject is determined to have no anomaly in the physical condition, the one or more features being extracted from among a plurality of features calculated as the feature per first period; and
generating, through supervised learning, a trained model that uses a feature of the subject as an input and outputs an anomaly score indicating a degree of an anomaly in the physical condition of the subject, the supervised learning using, as input data, the one or more features extracted, and using, as training data, the anomaly score corresponding to the one or more features extracted.

2. The trained model generation method according to claim 1, further comprising:
training, using the one or more features extracted, a data augmented model that outputs a feature corresponding to a case where the subject has no anomaly in the physical condition, wherein
the one or more features used as the input data include a feature generated by the data augmented model.

3. The trained model generation method according to claim 2, wherein
in the calculating of the feature per first period, a plurality of features are calculated per first period, and
when a two-dimensional arrangement of the plurality of features calculated is referred to as a feature set, the data augmented model outputs the feature set in which the plurality of features are two-dimensionally arranged.

4. The trained model generation method according to claim 3, wherein
a data generative model is used as the data augmented model.

5. The trained model generation method according to claim 3, wherein
one of a conditional generative adversarial network (CGAN), a variational autoencoder (VAE), an autoregressive model, or a diffusion model is used as the data augmented model.

6. The trained model generation method according to claim 1, further comprising:
obtaining the care record of the subject;
obtaining information indicating, in correspondence with the text data included in the care record, whether the subject has an anomaly in the physical condition; and
generating the natural language processing model through supervised learning that uses, as input data, the text data obtained, and uses, as training data, the information obtained that indicates whether the subject has an anomaly in the physical condition.

7. The trained model generation method according to claim 1, wherein
one of an anomaly detection with generative adversarial network (AnoGAN), a variational autoencoder (VAE), or a deep support vector data description (Deep SVDD) is used as the trained model.

8. The trained model generation method according to claim 1, wherein
the activity data includes at least one of food intake, a respiratory rate, a heart rate, a body temperature, or an out-of-bed rate of the subject.

9. The trained model generation method according to claim 1, wherein
the activity data includes at least two of food intake, a respiratory rate, a heart rate, a body temperature, or an out-of-bed rate of the subject.

10. A trained model generation device comprising:
a determiner that determines, per first period, whether a subject has an anomaly in a physical condition, based on a care record of the subject including text data, using a natural language processing model;
an obtainer that obtains activity data on the subject in a second period that includes a plurality of first periods each being the first period;
a calculator that calculates a feature per first period, based on the activity data obtained;
an extractor that extracts, based on a result of the determination, one or more features calculated for one or more first periods in which the subject is determined to have no anomaly in the physical condition, the one or more features being extracted from among a plurality of features calculated as the feature per first period; and
a generator that generates, through supervised learning, a trained model that uses a feature of the subject as an input and outputs an anomaly score indicating a degree of an anomaly in the physical condition of the subject, the supervised learning using, as input data, the one or more features extracted, and using, as training data, the anomaly score corresponding to the one or more features extracted.

11. A non-transitory computer-readable recording medium having recorded thereon a program for causing a computer to execute the trained model generation method according to claim 1.
